(19) United States
(12) Patent Application Publication
(10) Pub. No.: US 2022/0146482 A1
(43) Pub. Date: May 12, 2022

(54) ROOM-LEVEL INDOOR CO2 MEASUREMENT WITHOUT DEDICATED CO2 SENSORS

(71) Applicant: NEC Laboratories Europe GmbH, Heidelberg (DE)

(72) Inventors: Jonathan FUERST, Heidelberg (DE); Mauricio FADEL ARGERICH, Heidelberg (DE)

(21) Appl. No.: 17/182,319

(22) Filed: Feb. 23, 2021

Related U.S. Application Data
(60) Provisional application No. 63/112,726, filed on Nov. 12, 2020.

(51) Int. Cl. G01N 33/00; G06N 20/00; G06F 30/13

(52) U.S. Cl. CPC G01N 33/0062; G06F 30/13; G06N 20/00

(57) ABSTRACT

A method for measuring a CO₂ concentration inside a room includes extracting data from building sub-systems and integrating the data into a unified knowledge base and determining room layout. Occupancy of the room is predicted by applying information in the unified knowledge base to a set of knowledge functions. The CO₂ concentration inside the room is predicted by a CO₂ prediction model generated using the unified knowledge base, the determined room layout and the predicted occupancy of the room.

### CROSS-REFERENCE TO PRIOR APPLICATION

Priority is claimed to U.S. Provisional Patent Application No. 63/112,726 filed on Nov. 12, 2020, the entire disclosure of which is hereby incorporated by reference herein.

### FIELD

The present invention relates to a method, system and computer-readable medium for measuring room-level CO₂ levels without requiring dedicated CO₂ sensors using machine learning. The present invention can be integrated with a Building Management System (BMS), and also relates to such a BMS.

### BACKGROUND

Klepeis, Neil E., et al., “The National Human Activity Pattern Survey (NHAPS): a resource for assessing exposure to environmental pollutants,” Journal of Exposure Science & Environmental Epidemiology 11, no. 3, pp. 231-252 (2001) have found that people in developed countries spend most of their time (>90%) inside buildings, either in residential homes or in non-residential buildings such as their stores, office buildings, plants and other workplace. CO₂ levels have long been recognized to be an important indicator for “bad air” or insufficient ventilation inside buildings that makes people tired and uncomfortable, among other potential issues (see, e.g., Persily, Andrew, et al., “Carbon dioxide generation rates for building occupants,” Indoor air 27, no. 5, pp. 868-879 (2017)).

Therefore, sufficient building ventilation is necessary. To achieve sufficient ventilation, buildings have established natural ventilation mechanisms (e.g., windows), or automated ventilation mechanisms (e.g., though ventilation vents and a BMS-controlled Heating, Ventilation and Air-Conditioning (HVAC) systems). Most buildings do not have dedicated CO₂ sensors in every room (due to cost, deployment/integration/maintenance effort etc.), requiring to set the ventilation to a fixed setting (e.g., based on room size, maximum occupancy, following a building standard, such as those of the American Society of Heating, Refrigerating and Air-Conditioning Engineers (ASHRAE) or similar) or manually, based on actions by the occupants.

Recently, sufficient building ventilation has received even more importance, as SARS-CoV-2 is known to spread through aerosols, fine particles in the air that can stay in a space for extended amount of time without air-exchange.

To achieve adequate air exchange through ventilation, one solution is to use CO₂ levels from dedicated CO₂ sensors as an indication for when fresh air is needed. However, as mentioned above, such CO₂ sensors are costly and also present a number of other issues with respect to deployment and management, and with respect to integration into the technical systems of a building, such as the BMS and HVAC systems. In some buildings, it is even against the law or regulations to install sensors at certain locations. Another option is to provide for maximum ventilation. However, increased ventilation also results in high energy costs (it has been found that buildings already account for 40% of all energy use) and negative impact on human comfort (e.g., occupants are too cold, air gets too dry, etc.).

### SUMMARY

In an embodiment, the present invention provides a method for measuring a CO₂ concentration inside a room. The method includes extracting data from building sub-systems and integrating the data into a unified knowledge base and determining room layout. Occupancy of the room is predicted by applying information in the unified knowledge base to a set of knowledge functions. The CO₂ concentration inside the room is predicted by a CO₂ prediction model generated using the unified knowledge base, the determined room layout and the predicted occupancy of the room.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a system and method for room-level indoor CO₂ prediction without dedicated CO₂ sensors according to an embodiment of the present invention;

FIG. 2 schematically illustrates knowledge extraction into a unified knowledge base according to an embodiment of the present invention;

FIG. 3 schematically illustrates a method and system for providing a room/scene layout understanding according to an embodiment of the present invention;

FIG. 4 schematically illustrates the training of an occupancy prediction model according to an embodiment of the present invention;

FIG. 5 schematically illustrates a CO₂ prediction system in conjunction with a BMS according to an embodiment of the present invention; and

FIG. 6 schematically illustrates a CO₂ prediction system in conjunction with a mobile application according to an embodiment of the present invention.

### DETAILED DESCRIPTION

Embodiments of the present invention provide a method, system and computer-readable medium for room level-based CO₂ measurements without requiring dedicated and costly CO₂ sensors by accurate predictions using machine learning. This is achieved through building a data and knowledge driven machine learning model that processes available BMS sensor and actuator data, as well as data from other technical sub-systems of buildings such as the WiFi management system and the room booking system, and uses this data to learn a model that can predict accurate CO₂ level estimates on a per-room basis as relatively accurate room-level CO₂ measurements. By integrating with the technical systems of the building (e.g., the BMS, HVAC systems, WiFi management system, room booking system, etc.) and/or ubiquitous computer devices of occupants (e.g., laptops and smartphones), and using already-available data from those systems, embodiments of the present invention are able to provide room-level CO₂ measurements at low cost and hugely reduced technical overlay and maintenance, as dedicated CO₂ sensors are not needed in every room to provide such room-level CO₂ measurements. Moreover, embodiments of the present invention are easy to integrate into the existing technical systems of the building to improve the functioning of those systems to provide CO₂ measurements with a much higher granularity, or could be provided as new, improved systems. By providing for such room-level CO₂ measurements, embodiments of the present invention are able to save energy, and the corresponding costs, as well as reduce carbon emissions. Further, by providing for such room-level CO₂ measurements, embodiments of the present invention are able to provide for optimal levels of ventilation that allow buildings to operate more efficiently and safer. As used herein, the terms “measuring” and “measurement” also generally refers to a determination or prediction.

As already mentioned, dedicated CO₂ sensors are expensive and require significant effort and technical overlay for deployment, integration and maintenance. Embodiments of the present invention provide instead for virtual CO₂ sensors that do not require additional deployment effort or changes to existing buildings, but still are able to provide a meaningful input with room-level granularity for controlling building ventilation to keep occupants comfortable and safe.

Dedicated CO₂ sensors have been used to estimate building occupancy. In this context, state of the art systems have achieved ˜94% accuracy for a binary classification (room occupant vs not occupant) and ˜77% accuracy for counting the exact number of occupants (see Arief-Ang, et al., “SD-HOC: Seasonal decomposition algorithm for mining lagged time series,” In Australasian Conference on Data Mining, Springer, Singapore, pp. 125-143 (2017), which is hereby incorporated by reference herein). As such, there is a correlation between occupancy and CO₂ levels. A problem, however, is that this correlation is heavily skewed by the characteristics of different rooms (e.g., room volume, available windows, ventilation vents).

Embodiments of the present invention provide to use available building sensors and various data from sub-systems, such as the WiFi and room-booking system, together with room specific context features (i.e., room characteristics), derived automatically through automated computer-vision techniques (e.g., object recognition and scene understanding), to predict occupancy on a room-level and subsequently predict CO₂ levels on a per room-granularity in a knowledge and data-driven way using machine learning. Embodiments of the present invention are applicable and transferable between different buildings, room sizes, available sensors and installed ventilation mechanisms. Advantageously, embodiments of the present invention do not require the installation of additional infrastructure, such as dedicated CO₂ sensors.

In an embodiment, the present invention provides a method for measuring a CO₂ concentration inside a room. The method includes extracting data from building sub-systems and integrating the data into a unified knowledge base and determining room layout. Occupancy of the room is predicted by applying information in the unified knowledge base to a set of knowledge functions. The CO₂ concentration inside the room is predicted by a CO₂ prediction model generated using the unified knowledge base, the determined room layout and the predicted occupancy of the room.

In an embodiment, the method further comprises comparing the predicted CO₂ concentration to a predetermined CO₂ concentration for adjusting ventilation in the room.

In an embodiment, the method further comprises automated actuation of ventilation objects of the room using a server of a Building Management System (BMS).

In an embodiment, the method further comprises sending an electronic notification to a user device of an occupant of the room indicating that manual ventilation should occur.

In an embodiment, the data is transformed into a common format before being loaded into the unified knowledge base.

In an embodiment, the room layout is determined from building plans.

In an embodiment, the room layout is determined from pictures of the room using structure from motion and a room geometry estimation from a resulting point cloud, and using object recognition and localization techniques to identify and locate ventilation objects in the room.

In an embodiment, the set of knowledge functions are formed into an ensemble using a subset of labelled data.

In an embodiment, the method further comprises using the ensemble to label unlabeled data using an output of the ensemble.

In an embodiment, the subset of labelled data includes ground-truth from an existing CO₂ sensor.

In an embodiment, the method further comprises using ground-truth from an existing CO₂ sensor to calibrate the ensemble by assigning different weights to the knowledge functions based on the ground-truth.

In an embodiment, the method further comprises training an occupancy prediction model using the predicted occupancy.

In an embodiment, the method further comprises determining an activity level of an occupant of the room using a smart device of the occupant, and integrating the activity level into the unified knowledge base for the prediction of the CO₂ concentration.

In another embodiment, the present invention provides a system for measuring a CO₂ concentration inside a room, the system comprising one or more hardware processors configured, alone or in combination, to facilitate execution of the following steps: extracting data from building sub-systems and integrating the data into a unified knowledge base; determining room layout; predicting occupancy of the room by applying information in the unified knowledge base to a set of knowledge functions; and predicting the CO₂ concentration inside the room by a CO₂ prediction model generated using the unified knowledge base, the determined room layout and the predicted occupancy of the room.

In a further embodiment, the present invention provides a tangible, non-transitory computer-readable medium having instructions thereon, which, upon execution by one or more processors, provide for execution of the method according to an embodiment of the present invention.

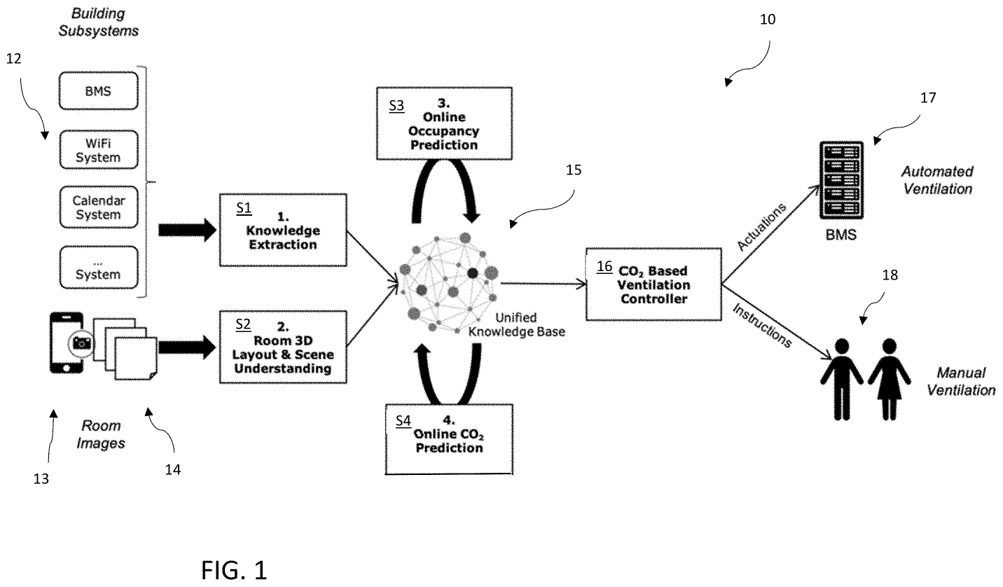

FIG. 1 schematically illustrates a method and system 10 for room-level indoor CO₂ prediction without dedicated CO₂ sensors according to an embodiment of the present invention. In a first step S1, knowledge extraction is performed from building subsystems 12, such as the BMS and any BMS servers, the WiFi system and the calendar system. In a second step S2 room 3D layout and scene understanding is provided using room images 14 from one or more smart devices 13, such as a smartphone, laptop or tablet. The outputs of these steps S1 and S2 are used as inputs into a unified knowledge base 15 which is used in step S3 to make an online occupancy prediction and in step S4 to make an online CO₂ prediction. The outputs of these steps S3 and S4 can be used for CO₂-based ventilation control 16 (e.g., through the automated or semi-automated actuation of parts of the HVAC systems using the servers of the BMS 17 or by providing instructions to users 18 on their devices). Each of these steps S1-S4 is discussed in further detail below.

### Step S1: Knowledge Extraction to Unify all Relevant Data Sources

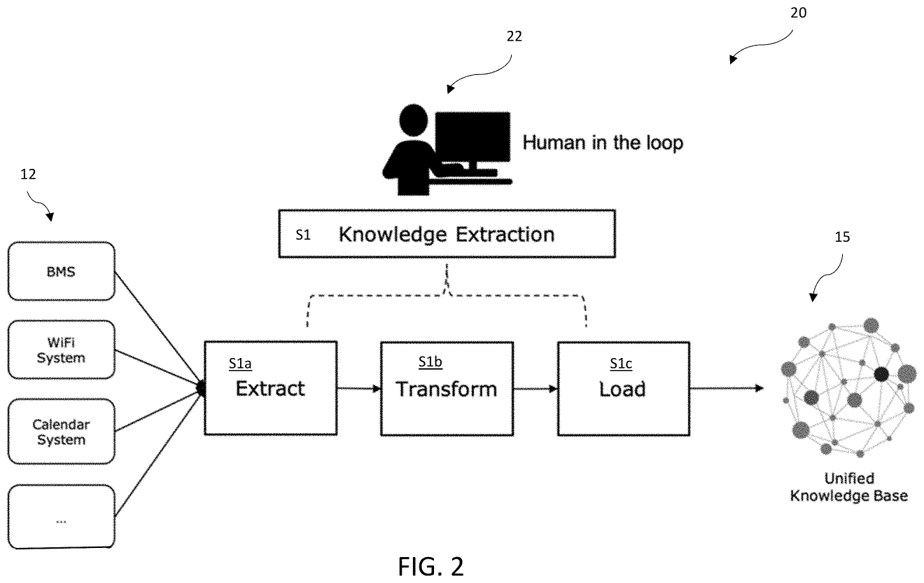

In an embodiment of the present invention illustrated in FIG. 2, a method and system 20 for the knowledge extraction step S1 relies on Artificial Intelligence (AI) supported Extract, Transform and Load (ETL) procedures to automate ingestion from semi-structured and structured data sources available in the various building sub-systems 12 (e.g., BMS, WiFi-System, digital calendars or facility management systems, etc.) into the unified knowledge base 15. In the Extract sub-step S1a, the data is extracted from the sub-systems. In the Transform sub-step S1b, the data is converted to a common format. In the Load sub-step S1c, the data is loaded into the unified knowledge base 15. This provides for the same semantic understanding of terms used in the different input sources, so that they can be more effectively integrated into a common knowledge graph to visualize their relationships. Existing tools and approaches for this integration/knowledge extraction step could also be used. First, data is extracted in Extract sub-step S1a by connecting to the building sub-systems 12 and retrieving data from, e.g., the BMS, or WiFi system. This data is then in semi-structured (e.g., json) or structured form (e.g., when connected to a relational DB). Second, in the Transform sub-step S1b, schema matching is applied to align the data schemas of the different extracted datasets. For example, if in one schema, there is a column labeled “temp” and in another schema of another dataset there is a column labeled “temperature”, it is necessary to detect that both columns represent temperature. Third, after having aligned schemas, it is possible to also perform entity matching. For example, the same meeting room might be described in the BMS system as in the WiFi system and the calendar system, and it is possible to detect with aligned schemas that it is the same room. After that, the data is integrated in the unified knowledge base 15 in the Load sub-step S1c. To automate these steps, there are various possibilities, such as “string similarities” and supervised ML techniques. Dong, Xin Luna, et al., “Big data integration,” Synthesis Lectures on Data Management 7, no. 1, pp. 1-198 (2015), which is hereby incorporated by reference herein, provide an overview of existing tools and ETL approaches. A human in the loop 22 can be used to define transformation/matching rules to transform the different data silos into a common data schema. It is also possible to oversee the knowledge extraction and, for example, remove clear outliers, filter the data and/or use ground-truth values from real CO₂ sensors to calibrate or adjust the data. The output of this step S1 is a unified, i.e., semantically understandable and integrated, database, e.g., in the form of a knowledge graph, or represented in other means (e.g., relational database).

### Step S2: Room 3D Layout/Scene Understanding

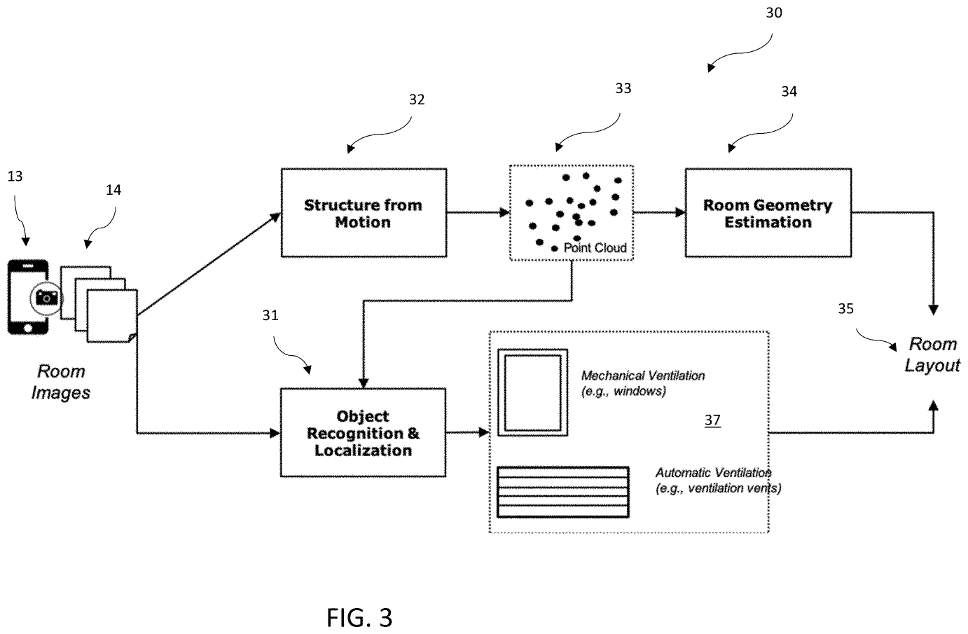

In an embodiment of the present invention illustrated in FIG. 3, a method and system 30 for the room 3D layout and scene understanding step S2 uses room images 14 (e.g., 2D pictures or images that are crowd-sourced from smartphone applications using smartphone cameras of one or more smart devices 13 of occupants or taken from the building's surveillance systems) as input for: (1) structure from motion (SfM) 32 and room geometry estimation 34 based on the resulting point cloud 33; and (2) object recognition and localization 31, wherein the object recognition is based on the room images 14 and the object localization is based on the room images 14 and the constructed point cloud 33 in order to achieve an understanding of the room layout 35 (i.e., the room geometry together with the relevant ventilation objects 37). SfM is discussed in Schonberger, Johannes L., et al., “Structure-from-motion revisited,” Proceedings of the IEEE Conference on Computer Vision and Pattern Recognition (2016), which is hereby incorporated by reference herein. Image localization is discussed in Chen, Kaifei, et al., “Snaplink: Fast and accurate vision-based appliance control in large commercial buildings,” Proceedings of the ACM on Interactive, Mobile, Wearable and Ubiquitous Technologies 1.4: 1-27 (2018), which is hereby incorporated by reference herein. Object recognition/detection is discussed in Redmon, Joseph, et al., “You only look once: Unified, real-time object detection,” Proceedings of the IEEE conference on computer vision and pattern recognition (2016), which is hereby incorporated by reference herein. The output of this step S2 is the room layout 35 as a room geometry together with available natural/mechanical and automated ventilation mechanisms and ventilation objects 37 (e.g., windows, doors and ventilation vents), integrated in the knowledge graph. Rather, than using 2D pictures, it would also be possible to manually enter the room layout into the system, e.g., based on available floor plans or knowledge of the facility management.

### Step S3: Online Occupancy Prediction

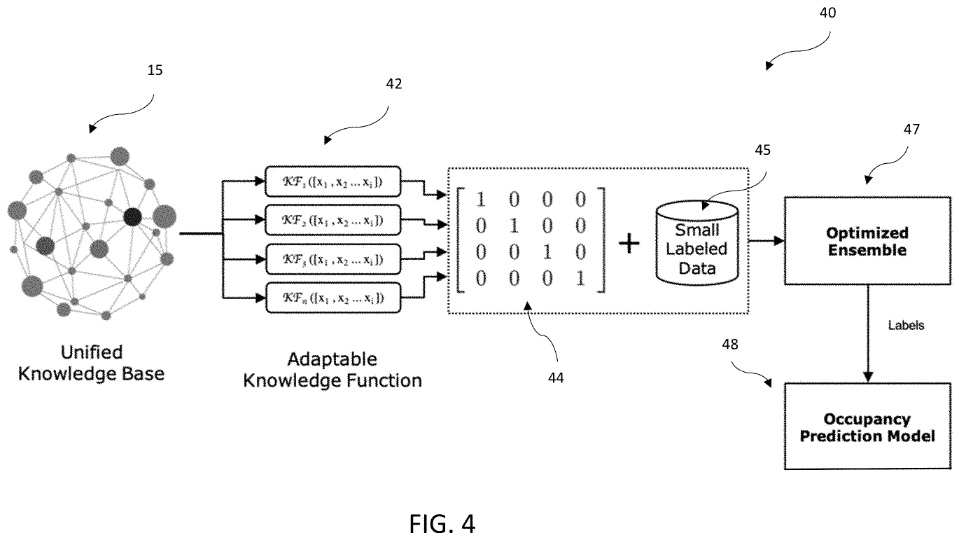

In an embodiment of the present invention illustrated in FIG. 4, a method and system 40 for arriving at an occupancy prediction model 48 from the information in the unified knowledge base 15. This occupancy prediction model 48 is constructed by utilizing all integrated data that is available in a flexible and adaptive fashion. Specifically, a set of knowledge functions 42 are constructed that access/query the knowledge base 15 and predict each individual room's occupancy based on some provided heuristics (e.g., number of devices connected to a specific access point, or calendar booking, etc.). If this data is not available, a function can abstain. Jointly, these knowledge functions 42 can be used to directly predict occupancy. The matrix 44 is based on the output of the knowledge functions 42. If there are N knowledge functions 42 and M data points, the matrix is N×M. An example of two knowledge functions 42 is provided in the following pseudocode. It is expected, however, that more than two knowledge functions would be used in most embodiments.

### Example:

```
def calendar_booking (outlook_room_booking):
  if outlook_room_booking = None:
    return 0
  else:
    return count(outlook_room_booking.get_attendees( ))
def wifi (access_point_in_room):
clients = access_point_in_room.get_clients( )
i = 0
for client in clients:
  if client.get_rssi( ) > −52 dBm:
    i=+1
return i
```

In this case, the first knowledge function 42 might output the value four (room has been booked and there are 4 attendees), while the second knowledge function 42 outputs zero (no clients connected). In an optimized ensemble 47 of such knowledge functions 42, it is possible to give more trust (and therefore weight) to the wifi-based function, even automatically using some small ground truth data. The output of the optimized ensemble 47 is the occupancy prediction. Accordingly, the optimized ensemble 47 is created from applicable knowledge functions 42, preferably together with a small set of collected labeled data 45 (e.g., occupancy levels in different rooms). It is possible to use the ensemble 47 to weakly label unlabeled data (e.g., data from various building sub-systems that have been integrated in the knowledge base 15). For example, data from the WiFi system, such as received signal strength indication (RSSI), is labeled with the ensemble output, e.g., the predicted occupancy. The resulting dataset of features and labels can be used to train a machine learning based occupancy prediction model 48. This trained occupancy prediction model 48 can then also be used for occupancy prediction with more generalizable and improved results compared to directly using the knowledge functions 42. As outputs of this step S3, the occupancy prediction model 48 is used to predict occupancy on a room level granularity. The unified knowledge base 15 is updated with the predictions.

In an embodiment of the present invention, the step S3 can also include a re-calibration of the method to a specific environment. If data from an existing, portable or old occupancy sensor (e.g., based on a CO₂ sensor, or based on a depth camera) is available for a room, this accurate occupancy sensing can be used to re-calibrate the ensemble 47 of knowledge functions 42 to further improve its accuracy. In other words, in step S3, the knowledge functions 42 have been joined into an ensemble 47 that assigns different weights to each knowledge function 42 based on different features, such as their correlation to other knowledge functions 42 and their accuracy. Correlation can be determined based on the output of multiple ones of the knowledge functions 42 across many data points and, based on this correlation, the weights can be adjusted. If accurate occupancy data for a room exists, this data can be used as ground-truth to re-calculate the weights of each knowledge function 42 into the ensemble 47, based on the empirical accuracy of the knowledge functions 42 when applied to the existing occupancy data. In this way, the ensemble 47 can be optimized for the current deployment and can provide a better accuracy, and then this ensemble 47 can be used to label new, unlabeled data that can later on be used to train a machine learning model. These results are integrated into the common unified knowledge base 15 for improving future models. Accordingly, labeled ground-truth data can be advantageously used to calibrate the ensemble of knowledge functions 42 (e.g., estimating the weight of each function based thereon). This ensemble 47 will then give more accurate occupancy predictions not only for the few rooms where there are labels, but also for all the rooms in the building.

### Step S4: Online CO₂ Prediction

With the occupancy prediction, the room layout understanding and preferably the BMS data integrated in the unified knowledge base 15, it is advantageously possible to now predict the CO₂ concentration on a room level granularity in accordance with embodiments of the present invention. For this prediction, this data is used as input into a physical CO₂ prediction model. Although the BMS data (e.g., a current ventilation rate) is not necessary, as it can be predicted, it can help to provide a more accurate prediction. In a space, the CO₂ behavior is defined as follows if volume of air stays constant (see Aglan, Heshmat A., “Predictive model for CO₂ generation and decay in building envelopes,” Journal of applied physics 93, no. 2, pp. 1287-1290 (2003), and Wang, Zhu, et al., “Indoor air quality control for energy-efficient buildings using CO 2 predictive model,” In IEEE 10th International Conference on Industrial Informatics, pp. 133-138 (2012), each of which is hereby incorporated by reference herein):

$$V \frac{d C ( t )}{d t} = q ( C ( t ) - {C}_{out} ( t ) ) + S ( t ) \qquad ( 1 )$$

where:
V is the volume of the space in m³
q is the ventilation rate in m³/s
Cₒᵤₜ is the outdoor CO₂ concentration in ppm
C is the indoor CO₂ concentration in ppm
S is the generation rate of CO₂

When it is considered that the air pressure does not change (i.e., that the inflow of air is always equal to the outflow), as common in buildings to avoid pressure differences, then the above equation can be written as follows, describing the generation and decay of CO₂ over time:

$$C ( t ) = ( {C}_{out} ( t ) + \frac{s ( t )}{q} ) - ( {C}_{o u t} ( {t}_{0} ) + \frac{s ( t )}{q} - C ( {t}_{0} ) ) {e}^{- q ( t - {t}_{0} ) / V} \qquad ( 2 )$$

In our invention, we substitute S with S=N G, where N are the number of occupants in a room and G represents the CO₂ generation rate per occupant. According to ASHRAE (American Society of Heating, Refrigerating and Air-Conditioning Engineers), this generation rate can be estimated by:

$${V}_{{CO}_{2}} = {V}_{{O}_{2}} R Q = \frac{0.00276 {A}_{D} M R Q}{( 0.23 RQ + 0.77 )} \qquad ( 3 )$$

where:
RQ is the ratio of the volumetric rate at which CO₂ is produced to the rate at which oxygen is consumed
M is level of physical activity, sometimes referred to as the metabolic rate, in units of met
A_{D} is the DuBois surface area (calculated from height and body mass)

Embodiments of the present invention can rely on calculating CO₂ generation with this equation based on occupant profiles. Occupant profiles might be obtained, e.g., from a profile saved in a smartphone or user device, or by using averages for different geographical regions. Another option is to use a fixed average value for CO₂ generation (available in ASHRAE standards) or use more accurate values for different sexes and age groups as published recently in Persily, Andrew, et al., “Carbon dioxide generation rates for building occupants,” Indoor air 27, no. 5, pp. 868-879 (2017), which is hereby incorporated by reference herein.

For Cₒᵤₜ, a configurable outdoor CO₂ concentration value is used that is set to 400 ppmv by default. If outside CO₂ levels are going to change in the future, this value can be updated. The value for V is derived from the room layout estimate (see step S2).

For the initial inside CO₂ concentration C(t₀), for a cold start of the system, the ventilation in the room is maximized (through BMS or natural ventilation) and then it can be safely assumed to use the same value as the outside CO₂ concentration for the inside CO₂ concentration (according to the mass balance method).

q is derived from the recognized ventilation objects, such as windows or ventilation vents, which can be combined (natural ventilation rate based on recognized windows, their size and geometry in the room, while the mechanical ventilation rate can be derived from BMS metadata or be estimated).

The output of step S4 is a virtual CO₂ level on a room granularity.

### CO₂ Based Ventilation Controller

The CO₂ based ventilation controller 16 actuates ventilation systems, preferably via servers of the BMS 17 based on the predicted CO₂ level and with a predetermined target CO₂ value that can be configured to ensure a comfortable working environment as well as safe conditions to avoid high densities of viral or bacterial aerosols (e.g., SARS-CoV-2). If available, the CO₂ based ventilation controller 16 utilizes the BMS 17 to actuate ventilation in an automated manner. Otherwise, it can notify occupants for when they should open the mechanical windows (e.g., through smartphone notifications or messages, email notifications, or through other means) to ensure natural ventilation. The result of this control is improved ventilation based on the online CO₂ prediction of step S4.

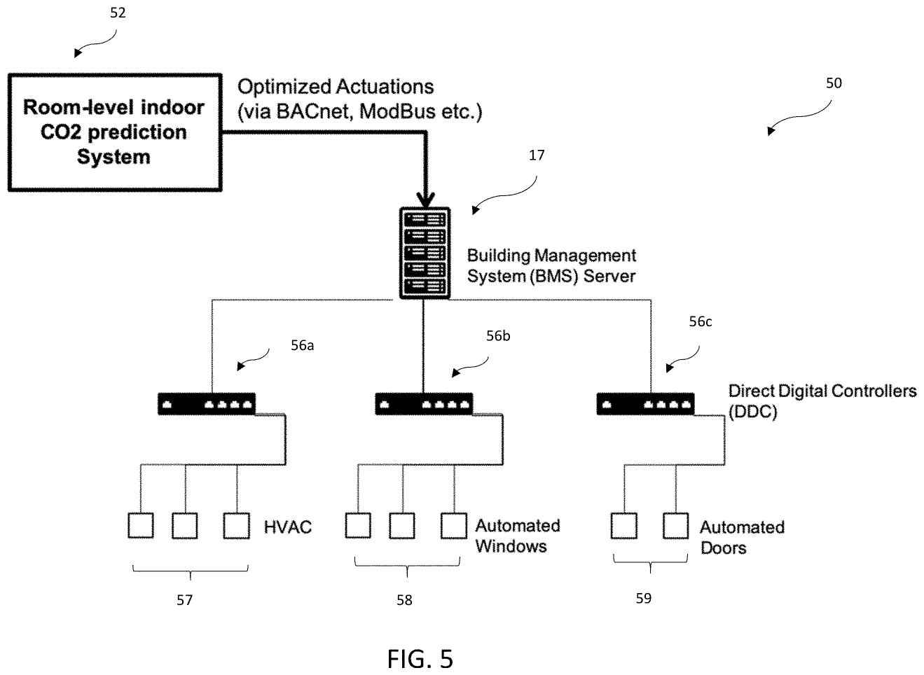

In an embodiment of the present invention illustrated in FIG. 5, based on the output of the CO₂ based ventilation controller 16 which is the output of the room-level indoor CO₂ prediction system 52, the BMS 17 is improved by being provided with optimized actuations through available networking interfaces (e.g., BACnet (a communication protocol for building actuation control) or ModBus (a communication protocol for programmable logic controllers)) to ensure adequate airflow in the building. The BMS 17, for example, actuates switches and or controllers (e.g., direct digital controllers (DDCs) 56a-c of respective HVAC systems 57 (e.g., automated ventilation devices), automated windows 58 and automated doors 59.

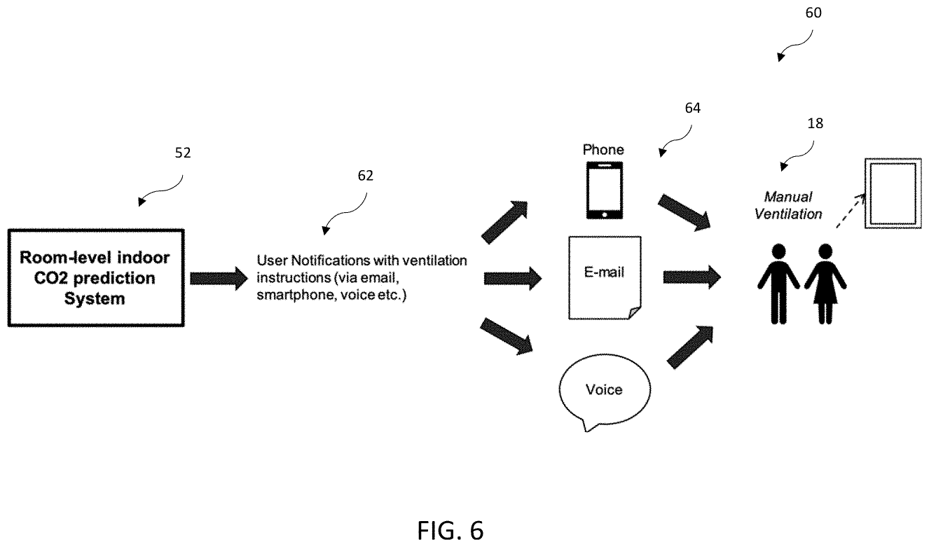

Alternatively or additionally, as illustrated in FIG. 6, a method and system 60 can be used to send users 18 who are occupants of the room in which the CO₂ prediction applies notifications 62 (e.g., via an application on their smartphones 64 or through e-mail) when they should open the windows and provide manual ventilation. Likewise, a later prediction can be used to notify users when it is safe to again close the windows.

In an embodiment of the present invention, the room-level indoor CO₂ prediction can be used for activity detection and occupant profiles. Recently, studies have shown that CO₂ generation rates of humans depend on human characteristics (e.g., body size, sex, different populations) and the current physical activity (see Persily, Andrew, et al., “Carbon dioxide generation rates for building occupants,” Indoor air 27, no. 5, pp. 868-879 (2017)). It is therefore possible in an embodiment of the present invention to include an activity detection module and the automated construction of building occupant profiles, to gain more accurate CO₂ predictions. Occupant activity detection can be performed with an application on the occupant's smartphones and potential smart wearables (e.g., smart watches) that utilizes smartphone activity recognition application programming interfaces (APIs) and available smartphone sensors to predict the current activity (e.g., standing, walking, sitting, etc.). This smartphone application can also be used to construct a user profile that includes the occupant's body size, sex, etc. This user profile can then be used in the online CO₂ prediction step S4 to obtain more accurate results.

Embodiments of the present invention enable the following improvements:
1. The creation of per-room occupancy prediction models in a flexible way that adapts automatically to the available data in the building through a dynamic and optimized ensemble of knowledge functions.
2. The physical modeling of CO₂ level behavior, without requiring CO₂ sensors, on a per room granularity using a combination of data from multiple building sub-systems and room occupancy together with room layout characteristics derived from multiple 2D images.

In an embodiment, the present invention provides a method for room-level CO₂ measurement and control comprising the steps of:
1. The virtual/physical integration of data from various building subsystems into a common knowledge base.
2. Obtaining the room layout (geometry and ventilation mechanisms), e.g., by collection of 2D images from a room from multiple angles and capturing relevant ventilation objects, specifically, through structure from motion and object recognition and localization (and integration of the results in the common knowledge base).
3. Occupancy prediction from the available information of the sub-systems integrated in the knowledge base, calculated through a set of knowledge functions. When ground-truth data is available for a room (e.g., from an existing CO₂ sensor, a small labelled dataset can be used to calibrate the ensemble of knowledge functions as described above. Further, a re-calibration of to a specific environment can also be performed as described above.
4. The creation of a physical room-level CO₂ prediction model utilizing the integrated knowledge base, the estimated room layout and the estimated real-time room occupancy.
5. The utilization of the predicted CO₂ to actuate the BMS optimally (e.g., using an air quality vs. energy consumption trade-off) or to notify building occupants to undertake necessary manual ventilation actions.

In contrast, current state of the art solutions require expensive and technically troublesome dedicated CO₂ sensors and/or perform building ventilation in a sub-optimal way (e.g., too little ventilation or too much ventilation and therefore too much energy use). Embodiments of the present invention can be used to provide a more efficient building ventilation with regards to energy savings (as discussed above, buildings are already a major energy consumer). Since building regulations require a minimum ventilation flow based on maximum occupancy of a room if sensor data for CO₂ or occupancy is not available, the result due to uncertainty is high energy costs (e.g., due to over-ventilation of rooms which may be empty). Thus, with respect to such situations, embodiments of the present invention can provide certainty and directly save energy and associated costs. Embodiments of the present invention can also be used to achieve a safer building operation with regards to aerosols and possible bacteria or virus levels. In the flu season or other pandemics, the air ventilation can be adjusted above the required amount with greater certainty with room-level granularity for thermal comfort and in order to reduce infections.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for measuring a CO₂ concentration inside a room, the method comprising:
extracting data from building sub-systems and integrating the data into a unified knowledge base;
determining room layout;
predicting occupancy of the room by applying information in the unified knowledge base to a set of knowledge functions; and
predicting the CO₂ concentration inside the room by a CO₂ prediction model generated using the unified knowledge base, the determined room layout and the predicted occupancy of the room.

2. The method according to claim 1, further comprising comparing the predicted CO₂ concentration to a predetermined CO₂ concentration for adjusting ventilation in the room.

3. The method according to claim 2, further comprising automated actuation of ventilation objects of the room using a server of a Building Management System (BMS).

4. The method according to claim 2, further comprising sending an electronic notification to a user device of an occupant of the room indicating that manual ventilation should occur.

5. The method according to claim 1, wherein the data is transformed into a common format before being loaded into the unified knowledge base.

6. The method according to claim 1, wherein the room layout is determined from building plans.

7. The method according to claim 1, wherein the room layout is determined from pictures of the room using structure from motion and a room geometry estimation from a resulting point cloud, and using object recognition and localization techniques to identify and locate ventilation objects in the room.

8. The method according to claim 1, wherein the set of knowledge functions are formed into an ensemble using a subset of labelled data.

9. The method according to claim 8, further comprising using the ensemble to label unlabeled data using an output of the ensemble.

10. The method according to claim 8, wherein the subset of labelled data includes ground-truth from an existing CO₂ sensor.

11. The method according to claim 8, further comprising using ground-truth from an existing CO₂ sensor to calibrate the ensemble by assigning different weights to the knowledge functions based on the ground-truth.

12. The method according to claim 1, further comprising training an occupancy prediction model using the predicted occupancy.

13. The method according to claim 1, further comprising determining an activity level of an occupant of the room using a smart device of the occupant, and integrating the activity level into the unified knowledge base for the prediction of the CO₂ concentration.

14. A system for measuring a CO₂ concentration inside a room, the system comprising one or more hardware processors configured, alone or in combination, to facilitate execution of the following steps:
extracting data from building sub-systems and integrating the data into a unified knowledge base;
determining room layout;
predicting occupancy of the room by applying information in the unified knowledge base to a set of knowledge functions; and
predicting the CO₂ concentration inside the room by a CO₂ prediction model generated using the unified knowledge base, the determined room layout and the predicted occupancy of the room.

15. A tangible, non-transitory computer-readable medium having instructions thereon, which, upon execution by one or more processors, provide for execution of the following steps:
extracting data from building sub-systems and integrating the data into a unified knowledge base;
determining room layout;
predicting occupancy of the room by applying information in the unified knowledge base to a set of knowledge functions; and
predicting the CO₂ concentration inside the room by a CO₂ prediction model generated using the unified knowledge base, the determined room layout and the predicted occupancy of the room.
